(19) United States
(12) Patent Application Publication
(10) Pub. No.: US 2025/0364287 A1
(43) Pub. Date: Nov. 27, 2025

(54) METHOD OF INSPECTING SEMICONDUCTOR PROCESS AND METHOD OF MANUFACTURING SEMICONDUCTOR DEVICE INCLUDING THE SAME

(71) Applicant: SAMSUNG ELECTRONICS CO., LTD., Suwon-si (KR)

(72) Inventors: Byeongseong AHN, Suwon-si (KR); Siho Song, Suwon-si (KR); Jiseong Doh, Suwon-si (KR); Jaehoon Jeong, Suwon-si (KR); Seunghyun Shin, Swuwon-si (KR)

(73) Assignee: SAMSUNG ELECTRONICS CO., LTD., Suwon-si (KR)

(21) Appl. No.: 18/956,617

(22) Filed: Nov. 22, 2024

(30) Foreign Application Priority Data
May 21, 2024 (KR) 10-2024-0065861

(51) Int. Cl. H01L 21/67; G01N 21/95; G01N 23/02; G01N 33/00; H01L 21/66

(52) U.S. Cl. CPC H01L 21/67288; G01N 21/9501; G01N 23/02; G01N 33/0095; H01L 22/12

(57) ABSTRACT

A method of inspecting a semiconductor process may include obtaining a measurement value of a semiconductor device, obtaining a simulation value of a virtual semiconductor device based on input data about a manufacturing process of the semiconductor device, wherein the input data may include a suspected defect-causing factor, comparing the measurement value with the simulation value, and providing information about a predicted defect-causing factor based on a comparison result between the measurement value and the simulation value.

### CROSS-REFERENCE TO RELATED APPLICATION

This application is based on and claims priority under 35 U.S.C. § 119 to Korean Patent Application No. 10-2024-0065861, filed on May 21, 2024, in the Korean Intellectual Property Office, the disclosure of which is incorporated by reference herein in its entirety.

### BACKGROUND

Apparatuses and methods consistent with example embodiments relate to inspecting a semiconductor process and manufacturing a semiconductor device using the same, and more particularly, to a method of inspecting a semiconductor process through machine learning and manufacturing a semiconductor device using the method of inspecting the semiconductor process.

Predicting the result by analyzing a semiconductor process in advance may improve the reliability of the characteristics of semiconductor devices and shorten the period for developing and researching semiconductor devices.

However, as semiconductor technology has recently become more advanced and more integrated, estimating and interpreting the process and results in advance by considering various conditions/factors of the processes may have great costs, such as time and computing resources. Accordingly, there is an increasing need for technologies for predicting the result of the semiconductor processes while providing improved explainability to users.

### SUMMARY

One or more embodiments include a method of inspecting a semiconductor process, whereby the semiconductor process may be precisely and effectively inspected, and a method of manufacturing a semiconductor device including the method of inspecting a semiconductor process.

According to an aspect of the present disclosure, a method of inspecting a semiconductor process may include: obtaining a measurement value of a semiconductor device; obtaining a simulation value of a virtual semiconductor device based on input data about a manufacturing process of the semiconductor device, wherein the input data may include a suspected defect-causing factor; comparing the measurement value with the simulation value; and providing information about a predicted defect-causing factor based on a comparison result between the measurement value and the simulation value.

According to another aspect of the present disclosure, a method of manufacturing a semiconductor process may include: obtaining a measurement value of a semiconductor device; obtaining a simulation value of a virtual semiconductor device based on input data about a manufacturing process of the semiconductor device, wherein the input data may include a suspected defect-causing factor; and comparing the measurement value with the simulation value by comparing similarity between an image of the semiconductor device that provides the measurement value and an image of the virtual semiconductor device that provides the simulation value.

According to another aspect of the present disclosure, a method of manufacturing a semiconductor device may include: preparing a wafer; forming a semiconductor device by performing a semiconductor process on the wafer; and inspecting the semiconductor process, wherein the inspecting of the semiconductor process may include: obtaining a measurement value of the semiconductor device; obtaining a simulation value of a virtual semiconductor device based on input data about a manufacturing process of the semiconductor device, wherein the input data may include a suspected defect-causing factor; comparing the measurement value with the simulation value; and providing information about a predicted defect-causing factor based on a comparison result between the measurement value and the simulation value.

### BRIEF DESCRIPTION OF DRAWINGS

The above and/or other aspects will be more apparent by describing certain example embodiments, with reference to the accompanying drawings, in which:

FIG. 1 is a view illustrating a semiconductor process inspection model according to one or more embodiments;

FIG. 2 is a block diagram illustrating a structure of a machine learning model according to one or more embodiments;

FIG. 3 is a view for describing a method of obtaining a measurement value, according to one or more embodiments;

FIG. 4 is a view for describing input data according to one or more embodiments;

FIG. 5 is a view illustrating a measurement value according to one or more embodiments;

FIG. 6 is a view illustrating a simulation value according to one or more embodiments;

FIG. 7 is a view illustrating comparison of a measurement value and a simulation value, according to one or more embodiments;

FIG. 8 is a flowchart illustrating a method of inspecting a semiconductor process, according to one or more embodiments;

FIG. 9 is a flowchart illustrating a method of manufacturing a semiconductor device, according to one or more embodiments;

FIG. 10 is a block diagram illustrating a processor providing a semiconductor process inspection apparatus, according to one or more embodiments; and

FIG. 11 is a flowchart illustrating a semiconductor process inspection apparatus according to one or more embodiments.

### DETAILED DESCRIPTION OF EMBODIMENTS

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

FIG. 1 is a view illustrating a semiconductor process inspection model according to one or more embodiments.

Referring to FIG. 1, a semiconductor process inspection model 10 may inspect a defect in a semiconductor process. The semiconductor process inspection model 10 may include process equipment 100 and a machine learning model 200.

The process equipment 100 may output an actual measurement value based on performing a semiconductor process to obtain inspection data by inspecting an actual semiconductor device. The process equipment 100 may also perform a process of manufacturing the semiconductor device, as well as performing the inspection process. In one or more embodiments, the measurement value may include numerical data obtained from the inspection data (e.g., an image of a semiconductor device, which may be captured by a camera mounted in or integrated into the process equipment 100 or received from an external device). For example, the measurement value may include a plan view, a cross-sectional view, a front view and/or a side view of the semiconductor device. For example, the process equipment 100 may include a high-resolution camera, an optical microscope (e.g., a brightfield microscope, a darkfield microscope), etc.), an optical measurement device, an electron microscope (e.g., a transmission electron microscope (TEM)), and/or a laser measuring instrument as measuring devices, and may use software for processing the measured data through an image imaging technology, an image processing technology, and/or a defect classification technology.

In another embodiment, the measurement value may include a shape, an arrangement, a width and/or a critical dimension (CD) of components of the semiconductor device. In another embodiment, the measurement value may be a value of a threshold voltage and/or the magnitude of a current of the manufactured semiconductor device.

However, the measurement value is not limited thereto and may include various values that represent characteristics of the semiconductor device. The measurement value may also mean data in which many actual results according to the previous processes are stored. For example, the measurement value may mean data obtained from a database (e.g., a domain knowledge database) in which actual process results are recorded.

The machine learning model 200 may output an estimated value that represents characteristics of the semiconductor device based on received input data. The input data for the machine learning model 200 may overlap with the input data for the process equipment 100. The overlapping input data may include process steps involved in manufacturing the semiconductor device, and defect-causing factors that may cause defects in the semiconductor device and which may be used to conduct the inspection process. The machine learning model 200 may output an output value that represents similarity between the measurement value and the estimated value. The machine learning model 200 may receive an actual measurement value obtained by the process equipment 100 for learning or a measurement value obtained from the database.

In one or more embodiments, the machine learning model 200 may include an artificial neural network (ANN) so as to inspect the semiconductor process. For example, the machine learning model 200 may include a long short-term memory (LSTM) technique ANN, which may refer to/include various computing systems based on a biological neural network that constitutes the animal brain. For example, the machine learning model 200 may include a convolutional neural network (CNN). However, the ANN included in the machine learning model 200 is not limited thereto and may be variously implemented. For example, the machine learning model 200 may also be implemented based on a gated recurrent unit (GRU) technique, an attention technique, or the like by including a recurrent neural network (RNN). In the ANN, performing of works may be learned by considering a plurality of samples (or examples) (e.g., a plurality of pieces of input data), unlike a classical algorithm that performs works according to pre-defined conditions, such as rule-based programming. The ANN may have a structure in which artificial neurons are connected to one another, and connection between the neurons may be referred to as a synapse. The neurons may process received signals and may transmit the processed signals to other neurons through the synapse. The output of the neurons may be referred to as activation. The neurons and/or synapse may have variable weights, and the effect of the signals processed by the neurons may be increased or decreased according to the weights. In particular, the weights related to individual neurons may be referred to as bias.

However, for a machine learning method for inspecting a semiconductor process, the machine learning model 200 is not limited to being based on the ANN but may be implemented based on various learning methods and/or algorithms. For example, the machine learning model 200 may also be implemented based on a random forest technique.

The input data may include information used to define sub-process steps (hereinafter, referred to as process steps) involved in manufacturing the semiconductor device being inspected by the process equipment 100, and may include a defect-causing factor that may cause defects in the semiconductor device. The sub-process steps may refer to process steps for manufacturing the semiconductor device. For example, the sub-process steps may mean a photolithography process, an etching process, an ion implantation process, a planarization process such as chemical mechanical polishing (CMP), and the like.

The defect-causing factor may include all factors that may cause a defect in a semiconductor process. For example, the defect-causing factor may include particles. For example, the defect-causing factor may include an error (e.g., over or under-progress of an etching process) of the semiconductor process.

The input data may include physical characteristics of the defect-causing factor and a time of occurrence of the defect-causing factor. For example, the physical characteristics of the defect-causing factor may include the shape of the defect-causing factor, the position of the defect-causing factor, the size of the defect-causing factor and/or the property of the defect-causing factor. Here, the property of the defect-causing factor may refer to a function (or action) of the defect-causing factor in the semiconductor process. For example, the defect-causing factor may act as a type of photoresist in a specific process step, and may act as a negative tone photoresist or a positive tone photoresist according to chemical characteristics of the defect-causing factor.

The machine learning model 200 may provide or output an estimated value based on received input data, as will be described below in detail. The estimated value may represent a predication of a measurement value (e.g. a value for predicting the result of a measurement operation). The estimated value may be a value for predicting the characteristics (e.g., the arrangement of components etc.) of the semiconductor device to be manufactured by sub-process steps of the input data. The estimated value may include a value for predicting a change in the characteristics of the semiconductor device according to a change in physical characteristics. In one or more embodiments, the machine learning model 200 may output an estimated value including a plan view, a front view, a side view and/or a cross-sectional view of the semiconductor device. The estimated value may undergo further processing within the machine learning model 200 to provide an output value that represents the similarity between the measurement value and the estimated value, as detailed below.

Here, a measurement operation may refer to an operation of checking whether one or more sub-process steps are properly performed. For example, the measurement operation may refer to an operation of testing the structure and/or electrical characteristics of the semiconductor device after a series of sub-process steps are performed.

In addition, the machine learning model 200 may output an output value based on the measurement value and the estimated value. The output value may be a value representing similarity between the measurement value and the estimated value. Based on the output value, the cause of the defect that has occurred in the semiconductor process may be easily identified. In other words, if some (e.g., a defect-causing factor) of the conditions of the sub-process steps change, the characteristic (e.g., the arrangement of components) of the device that change accordingly may be a predicted value.

Thus, the semiconductor process inspection model 10 may provide a method of easily grasping the semiconductor process in which physical characteristics of the defect-causing factor and/or the defect-causing factor occurs (or is input) when the semiconductor device acquired from the process equipment 100 has a defect.| Thus, the semiconductor process inspection model 10 may remove a defect in the semiconductor process rapidly and precisely.

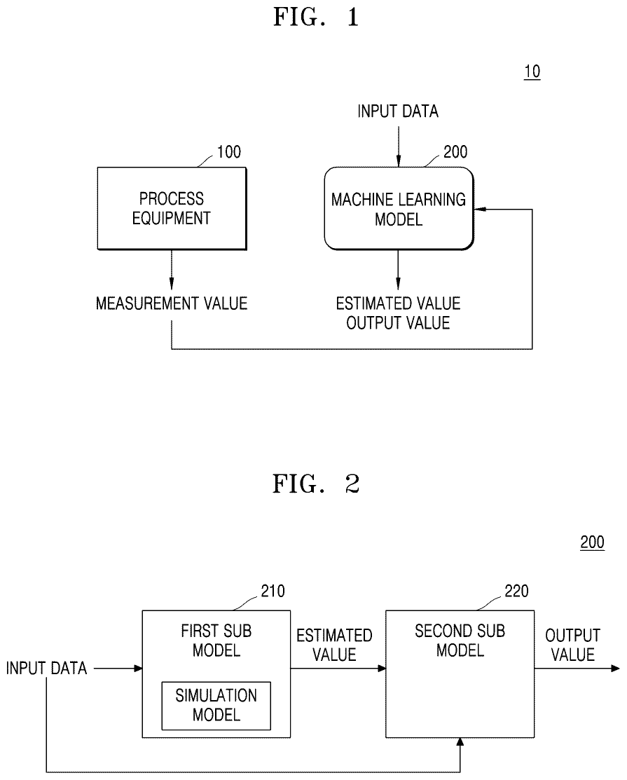

FIG. 2 is a block diagram illustrating the structure of a machine learning model according to one or more embodiments.

Referring to FIG. 2, the machine learning model 200 may include a first sub-model 210 and a second sub-model 220. Input data input to the machine learning model 200 may be grouped (or classified), and the machine learning model 200 may output an estimated value and an output value, as described above, based on the grouped input data.

In one or more embodiments, the first sub-model 210 may include a simulation model. The first sub-model 210 may perform modeling of the semiconductor device manufacturing process. For example, the simulation model may include technology computer-aided design (TCAD). In one or more embodiments, the TCAD may simulate the semiconductor process to provide information about a virtual semiconductor device. However, the technical spirit of the present disclosure is not limited thereto, and any simulation module capable of modeling the semiconductor process may be used.

The first sub-model 210 may obtain information about the semiconductor device manufactured in various conditions. For example, the first sub-model 210 may obtain information about a simulation semiconductor device based on the physical characteristics of the defect-causing factor (e.g., the shape of the defect-causing factor, the position of the defect-causing factor, the size of the defect-causing factor and/or the property of the defect-causing factor) and/or a time of occurrence of the defect-causing factor. In detail, the first sub-model 210 may simulate (or estimate) a defect with a specific shape, such as a circular void or a crack in the semiconductor material. In this case, the estimated value may represent the dimensions of the defects dimensions and how the defects affect electrical conductivity. The first sub-model 210 may simulate (or estimate) defects occurring at various positions on the semiconductor wafer, such as near critical circuit paths. The estimated value may include data on how the position impacts the overall performance of the semiconductor device. The first sub-model 210 may simulate (or estimate) defects of different sizes, such as microscopic particles. The estimated value may quantify the impact of these sizes on yield and reliability. The first sub-model 210 may simulate (or estimate) defects with different material properties, such as conductivity or thermal resistance, and the estimated value may reflect how these properties influence device functionality. The first sub-model 210 might simulate (or estimate) defects based on the timing of their occurrence during manufacturing, such as during specific process steps. The estimated value may indicate how the timing correlates with the likelihood of defects affecting device performance.

In one or more embodiments, the second sub-model 220 may compare the measurement value with the estimated value. For convenience of explanation, the estimated value (i.e., the information about the simulation semiconductor device) obtained by the first sub-model 210 may refer to a simulation value. That is, the second sub-model 220 may compare the estimated value with the simulation value. The second sub-model 220 may perform various operations based on the estimated value and the simulation value and may output an output value as the result of performing. In one or more embodiments, the second sub-model 220 may perform an arithmetic operation on the measurement value and the simulation value based on an artificial neural network. In one or more embodiments, the second sub-model 220 may output similarity between the measurement value and the estimated value.

However, the machine learning model 200 is not limited to the present embodiment but may further include various sub-models for performing several functions. The first sub-model 210 and/or the second sub-model 220 may also include lower sub-model(s) therein.

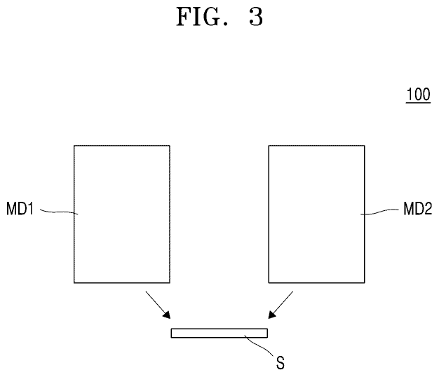

FIG. 3 is a view for describing a method of obtaining a measurement value according to one or more embodiments. This will be described with reference to FIG. 1 together.

Referring to FIG. 3, the process equipment 100 may include a first measurement device MD1 and a second measurement device MD2. The first measurement device MD1 and the second measurement device MD2 may inspect an object S (e.g., a semiconductor device) to obtain data about the arrangement of components inside the object S. In one or more embodiments, the first measurement device MD1 may obtain the plan view of the object S, and the second measurement device MD2 may obtain the cross-sectional view of the object S. As described above, a result obtained by the first measurement device MD1 and the second measurement device MD2 of the process equipment 100 may be referred to as a measurement value. In FIG. 3, obtaining of the plan view and the cross-sectional view of the object S is exemplarily described. However, the technical spirit of the present disclosure is not limited thereto. For example, the measurement device may measure the front view and/or the side view of the object S, or may obtain various images for inspecting the object S.

In one or more embodiments, the first measurement device MD1 may include an optical measurement device, and the second measurement device MD2 may include a transmission electron microscope (TEM). For example, the first measurement device MD1 may include a brightfield microscope and/or a darkfield microscope. In one or more embodiments, the first measurement device MD1 may obtain data about the object S using a non-destructive inspection method, and the second measurement device MD2 may obtain data about the object S using a destructive inspection method. However, the technical spirit of the present disclosure is not limited thereto, and any equipment capable of measuring various data about the object S may be included in the measurement device.

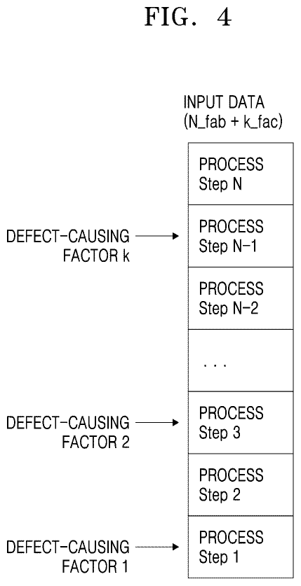

FIG. 4 is a view for describing input data according to one or more embodiments. This will be described with reference to FIGS. 1 and 2 together.

Referring to FIG. 4, the input data may be data for defining N sub-process steps and k defect-causing factors (where N and k are natural numbers of 2 or more) and may be provided to the machine learning model 200. For example, the defect-causing factor may be generated (or input) in an i-th process step or may be generated (or input) in a j-th process step (where i and j are natural numbers of 1 or more). Also, the physical characteristics of the defect-causing factor input in each process step may be variously changed. For example, the physical characteristics of the defect-causing factor may include the shape of the defect-causing factor, the position of the defect-causing factor, the size of the defect-causing factor and/or the property of the defect-causing factor. The defect-causing factor may include a plurality of factors. The first sub-model 210 may obtain a simulation value based on the physical characteristics of the defect-causing factor and/or a time of occurrence of the defect-causing factor.

Thus, in the method of inspecting the semiconductor device of the present disclosure, a simulation value in various conditions may be obtained, and the estimated value and the simulation value may be easily compared with each other. Thus, in the method of inspecting the semiconductor device of the present disclosure, the physical characteristics of the defect-causing factor and/or the time of occurrence of the defect-causing factor may be rapidly and precisely inspected.

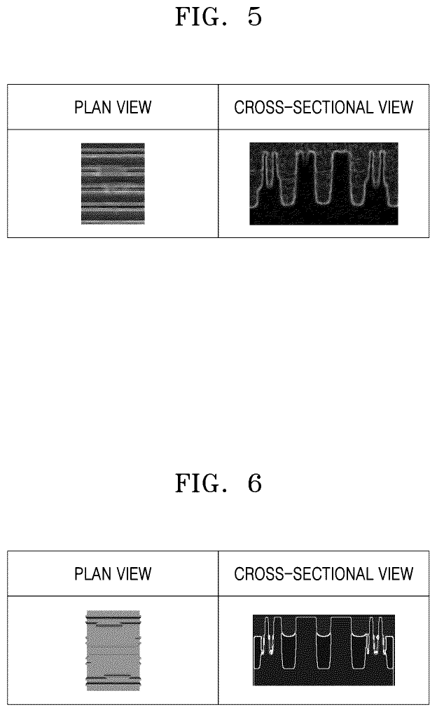

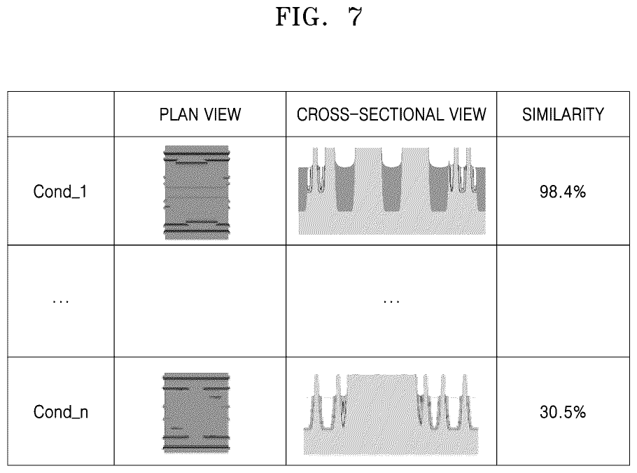

FIG. 5 is a view illustrating a measurement value according to one or more embodiments, and FIG. 6 is a view illustrating a simulation value according to one or more embodiments. FIG. 7 is a view illustrating comparison of a measurement value and a simulation value according to one or more embodiments. This will be described with reference to FIGS. 1 through 4 together.

Referring to FIGS. 5 through 7, the process equipment 100 may obtain a measurement value by measuring the semiconductor device. In addition, the first sub-model 210 may obtain a simulation value based on the simulation model. The first sub-model 210 may obtain a plurality of simulation values according to various conditions (the physical characteristics of the defect-causing factor and/or a time of occurrence of the defect-causing factor). FIG. 6 illustrates the plan view and the cross-sectional view of one simulation value exemplarily, but a plurality of simulation values may be obtained. The measurement value and the simulation value may include the plan view and the cross-sectional view of the semiconductor device.

In one or more embodiments, the first sub-model 210 may obtain a plurality of simulation values according to various conditions (the physical characteristics of the defect-causing factor and/or the time of occurrence of the defect-causing factor) in a specific process. The specific process may be a suspected process. The suspected process may mean a process in which it is predicted that a defect occurs.

The second sub-model 220 may compare the estimated value with the simulation value to calculate similarity between the measurement value and the simulation value. In one or more embodiments, the second sub-model 220 may calculate similarity between the measurement value and the simulation value using the artificial neural network. In one or more embodiments, the second sub-model 220 may calculate similarity between the measurement value and the simulation value using the convolutional neural network.

In one or more embodiments, the second sub-model 220 may compare simulation values generated in a plurality of conditions, with a measurement value. FIG. 7 illustrates comparison of simulation values generated in n conditions (where n is a natural number of 1 or more) with the measurement value. The second sub-model 220 may compare each of the simulation values generated in a plurality of conditions with the measurement value to align each of the simulation values in an order of high similarity with the measurement value. Based on the condition of the simulation value having the highest similarity, the defect of the semiconductor process may be easily grasped. Based on the simulation value having the highest similarity to the measurement value obtained from the process equipment 100, the physical characteristics of the defect-causing factor of the process equipment 100 and/or the time of occurrence of the defect-causing factor may be determined. For example, when the first condition (Cond_1) used to obtain the simulation value shows the highest similarity (e.g., 98.4%) to the measurement value compared to other conditions (Cond_1, Cond_2, . . . , Cond_n), the physical characteristics of the defect-causing factor in the process equipment 100 and/or the timing of its occurrence under this first condition (Cond_1) may be considered as contributing factors to the defects. The different conditions may be understood as different defect-causing factors suspected of causing defects in the semiconductor device. These defect-causing factors may be referred to as suspected defect-causing factors, and the second sub-model 220 may provide at least one predicted defect-causing factor, which has the highest similarity, among the suspected defect-causing factors.

In addition, in order to easily compare the measurement value with the simulation value, the boundary of an image of each of the measurement value and the simulation value may be detected. In more detail, the boundary between components of the image of the measurement value obtained by the process equipment 100 may be detected, and the boundary between components of the image of the simulation value obtained by the first sub-model 210 may be detected. Boundary detection may be performed by the process equipment 100, the first sub-model 210 and/or the second sub-model 220.

For example, the boundary detection may be performed in a gradient filter manner. In the gradient filter method, a point at which an image is changed into a gray scale and then the gray scale rapidly changes, may be determined as a boundary. However, the technical spirit of the present disclosure is not limited thereto, and a different method of detecting the boundary of an image may also be used.

FIGS. 5 and 6 illustrate images in which boundary detection is performed. However, embodiments of the present disclosure are not limited thereto, and the process equipment 100 and/or the first sub-model 210 may obtain an image in which boundary detection is not performed. In addition, the second sub-model 220 may compare the estimated value and the simulation value with each other based on images in which boundary detection is not performed.

In addition, the second sub-model 220 may perform learning based on the measurement value and the simulation value. The second sub-model 220 may update a model for determining similarity by performed learning.

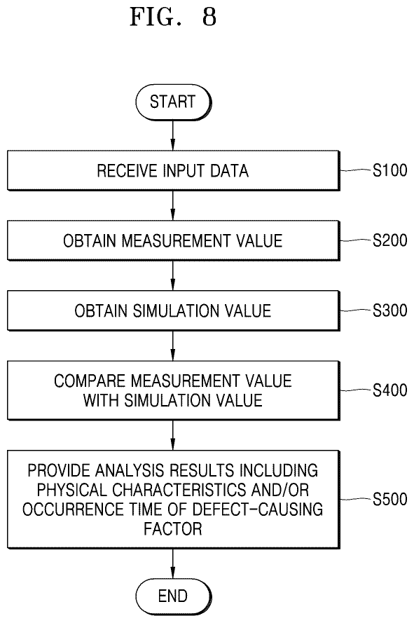

FIG. 8 is a flowchart illustrating a semiconductor process inspection model according to one or more embodiments. This will be described with reference to FIGS. 1 through 7 together.

Referring to FIG. 8, first, input data may be received (S100). Here, the input data may include a plurality of pieces of semiconductor process data and defect-causing factor data. Here, the defect-causing factor data may include the physical characteristics of the defect-causing factor and/or the time of occurrence of the defect-causing factor. For example, the physical characteristics of the defect-causing factor may include the shape of the defect-causing factor, the size of the defect-causing factor, the position of the defect-causing factor and/or the property of the defect-causing factor.

Thereafter, the process equipment 100 may obtain a measurement value (S200). The process equipment 100 may output a measurement value of the semiconductor device obtained by performing the semiconductor process. The process equipment 100 may obtain a measurement value based on the plan view, the front view, the side view and/or the cross-sectional view of the semiconductor device.

Thereafter, the machine learning model 200 may obtain the simulation value (S300). The first sub-model 210 of the machine learning model 200 may obtain the simulation value of the semiconductor device in various conditions based on the simulation model. For example, the first sub-model 210 may include TCAD.

In one or more embodiments, the first sub-model 210 may perform simulation by inputting the defect-causing factor in a process (a suspected process) in which it is predicted that a defect occurs, based on the measurement value of the process equipment 100. In another embodiment, the first sub-model 210 may perform simulation in each of various conditions (the physical characteristics of the defect-causing factor and/or the time of occurrence of the defect-causing factor).

Thereafter, the machine learning model 200 may determine the physical characteristics of the defect-causing factor and/or the time of occurrence of the defect-causing factor by comparing the measurement value with the simulation value (S400). In one or more embodiments, the second sub-model 220 of the machine learning model 200 may compare the measurement value with the simulation value, for example, using the artificial neural network. In one or more embodiments, the second sub-model 220 may compare an image of the measurement value with an image of the estimated value. The second sub-model 220 may calculate similarity between the image of the measurement value and the image of the estimated value. The similarity may be measured by using a cosine similarity function or an Euclidean distance function. For example, the second sub-model 220 may include a convolutional neural network. In more detail, the second sub-model 220 may determine the image of the simulation value having a highest similarity to the image of the measurement value obtained by the process equipment 100 and then, may determine the physical characteristics of the defect-causing factor and/or the time of occurrence of the defect-causing factor based on the image of the simulation value.

The semiconductor process inspection model 10 may provide an analysis result including the physical characteristics of the defect-causing factor and/or the time of the occurrence of the defect-causing factor (S500).|

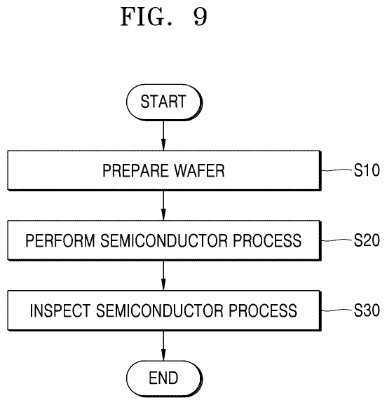

FIG. 9 is a flowchart illustrating a method of manufacturing a semiconductor device according to one or more embodiments. This will be described with reference to FIGS. 1 through 8 together.

First, a wafer may be prepared (S10). The wafer may include a bare wafer in which one or more semiconductor processes are performed or a semiconductor process is not performed, for example.

Thereafter, the semiconductor process may be performed on the wafer (S20). An oxidation process, a photolithography process, a deposition process, an etching process, an ion implantation process and/or a cleaning process, and the like may be performed on the wafer. The semiconductor process may include a simulation process of individualizing the wafer into each of semiconductor chips, a test process of testing the semiconductor chips, and a packaging process of packaging the semiconductor chips. A semiconductor device may be completed through the semiconductor process on the wafer.

Thereafter, semiconductor process inspection may be performed (S30). As shown in FIG. 8, the semiconductor process inspection (S30) may include receiving input data (S100), obtaining a measurement value (S200), obtaining a simulation value (S300), and comparing the measurement value with the simulation value (S400). In Operation S30, Operations S100 through S400 of FIG. 8 may be performed to detect a defect of the semiconductor process of manufacturing the semiconductor device when a defect occurs in the semiconductor device.

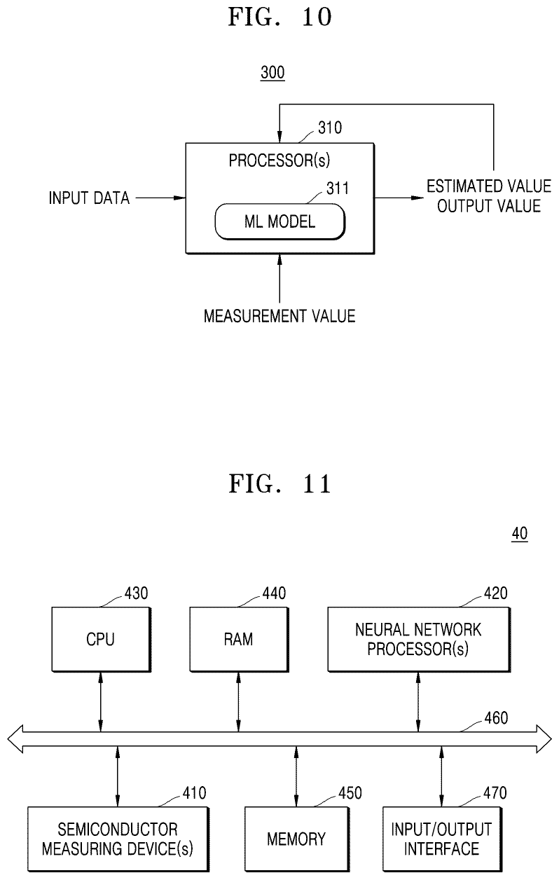

FIG. 10 is a block diagram illustrating a processor providing a semiconductor process inspection apparatus according to one or more embodiments. This will be described with reference to FIG. 1 together.

Referring to FIG. 10, at least one processor 310 may provide a machine learning model 311 for inspecting the semiconductor process. The machine learning model 311 of FIG. 10 may correspond to the machine learning model 200 of FIG. 1.

At least one processor 310 may execute a program module including system executable command languages. The program module may include routines, programs, objects, components, logic, data structures, and the like, for performing a specific task or implementing a specific abstract data type. For example, at least one processor 310 may include one or more of a central processing unit (CPU), a graphics processing unit (GPU), and a neural processing unit (NPU). However, this is an example, and at least one processor 310 is not limited to the description above. At least one processor 310 may receive input data for defining sub-process steps and defect-causing factors. At least one processor 310 may execute the machine learning model 311 to output an estimated value that represents the characteristics of the semiconductor device. At least one processor 310 may output an output value that represents similarity between input data and an estimated value based on the input data and the estimated value. In addition, at least one processor 310 may receive a measurement value that is actually measured by performing a process to train the machine learning model 311 and/or a measurement value obtained from a database.

In some embodiments, the machine learning model 311 may include an artificial neural network and/or an algorithm so as to inspect a semiconductor process. The machine learning model 311 may output the estimated value based on the input data. The estimated value may be a value for predicting the result of the semiconductor process including defect-causing factors, and the machine learning model 311 may output the estimated value by predicting the result of manufacturing the semiconductor device according to the physical characteristics of the defect-causing factor and/or the time of occurrence of the defect-causing factor. The machine learning model 311 may output the output value by determining similarity between the measurement value and the simulation value based on the measurement value and the simulation value. The output value of the machine learning model 311 may include a value for calculating similarity between the measurement value and the estimated value.

FIG. 11 is a flowchart illustrating a semiconductor process inspection apparatus according to one or more embodiments. This will be described with reference to FIGS. 1 through 10 together.

Referring to FIG. 11, a semiconductor process inspection apparatus 40 may obtain a measurement value and a simulation image and may inspect a semiconductor process by comparing the measurement value with the simulation image using a neural network. The semiconductor process inspection apparatus 40 may include a semiconductor device measurement apparatus 410, a neural network processor 420, a CPU 430, a random access memory 440, a memory 450, a bus 460, and an input/output interface 470. The input/output interface 470 may include an input interface such as a physical keyboard, a virtual keyboard, a touchpad, and a microphone to receive various input data (e.g., defector-causing factors 1, 2, . . . k). The input/output interface 470 may include an output interface such as a display and a speaker. For example, the output interface may display the images and the similarity values shown in FIGS. 5, 6, and 7, on the display. Additionally, the output interface may also display a plurality of process steps 1, 2, 3, . . . , N−2, N−1, and N and the corresponding defector-causing factors 1, 2, . . . k. The input/output interface 470 may also include a communication interface to receive the input data or transmit the output value of the semiconductor process inspection model 10. The communication interface may be implemented by any one or any combination of a digital modem, a radio frequency (RF) modem, an antenna circuit, a WiFi chip, and related software and/or firmware.

According to one or more embodiments, the semiconductor process inspection apparatus 40 may further include other general-purpose components in addition to the components shown in FIG. 11. For example, the semiconductor process inspection apparatus 40 may further include an input/output module, a security module, a power control device, and the like, and may further include various types of processors. In addition, according to one or more embodiments, at least one of the components of FIG. 11 may be omitted from the semiconductor process inspection apparatus 40. The components of the semiconductor process inspection apparatus 40 may communicate with each other through the bus 460.

The semiconductor device measurement apparatus 410 may inspect the semiconductor device to acquire an image of the semiconductor device. In one or more embodiments, the semiconductor device measurement apparatus 410 may acquire a plan view, a front view, a side view, and/or a cross-sectional view of the semiconductor device. For example, the semiconductor device measurement apparatus 410 may include an optical-based measurement apparatus and/or a transmission electron microscope (TEM).

The neural network processor 420 may infer information included in the input data by training (or learning) the neural network or analyzing the input data using the neural network. The neural network processor 420 may determine a situation based on the inferred information or control components of a mounted electronic device.

In addition, the neural network processor 420 may receive input data from the semiconductor device measurement apparatus 410 and the memory 450 and may generate output data based on the received input data. The neural network processor 420 may obtain a simulation value by performing simulation based on the physical characteristics of the defect-causing factor and/or the time of occurrence of the defect-causing factor. The neural network processor 420 may compare the estimated value with the simulation value to calculate similarity between the measurement value and the simulation value.

In another embodiment, the neural network processor 420 may further include an additional processor, and the processor may obtain a simulation value by performing a simulation based on the physical characteristics of the defect-causing factor and/or the time of occurrence of the defect-causing factor.

The neural network processor 420 may be implemented as a neural network operation accelerator, a coprocessor, a digital signal processor (DSP), an application specific integrated circuit (ASIC), a field-programmable gate array (FPGA), a graphics processing unit (GPU), a neural processing unit (NPU), a sensor processing unit (TPU), and a multi-processor system-on-chip (MPoSC).

The neural network processor 420 may be a neural network model based on an artificial neural network (ANN), a convolution neural network (CNN), a region with convolution neural network (R-CNN), a region proposal network (RPN), a recurrent neural network (RNN), a generative adversarial network (GAN), a self-attention generative adversarial network (SAGAN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) network, a classification network, a plain residual network, a dense network, a hierarchical pyramid network, a transformer and/or vision transformer network. Types of neural network models are not limited to the above-described examples.

The CPU 430 may control the general operation of the semiconductor process inspection apparatus 40. The CPU 430 may include a single processor core or a plurality of processor cores. The CPU 430 may process or execute programs and/or pieces of data stored in a storage region such as the memory 450 using a RAM 440.

For example, the CPU 430 may execute an application and control the neural network processor 420 to perform neural network-based tasks required according to the execution of the application program.

The memory 450 may store a measurement value and/or a simulation value. The measurement value and/or the simulation value may include an image. In one or more embodiments, the measurement value and/or the simulation value may include a plan view, a front view, a side view and/or a cross-sectional view of a semiconductor device.

The memory 450 may include at least one of a volatile memory and a nonvolatile memory. The nonvolatile memory includes a read only memory (ROM), a programmable ROM (PROM), an electrically programmable ROM (EPROM), an electrically erasable and programmable ROM (EEPROM), and a flash memory. The volatile memory may include a dynamic RAM (DRAM), a static RAM (SRAM), a synchronous DRAM (SDRAM), a phase-change RAM (PRAM), a magnetic RAM (MRAM), a resistive RAM (RRAM), a ferroelectric RAM (FeRAM), and the like. In embodiments, the memory 160 may include at least one of a hard disk drive (HDD), a solid state drive (SSD), a compact flash (CF) card, a secure digital (SD) card, a micro-secure digital (Micro-SD) card, a mini-secure digital (mini-SD) card, an extreme digital (xD) card, or a memory stick.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A method of inspecting a semiconductor process, the method comprising:
obtaining a measurement value of a semiconductor device;
obtaining a simulation value of a virtual semiconductor device based on input data about a manufacturing process of the semiconductor device, wherein the input data comprises a suspected defect-causing factor;
comparing the measurement value with the simulation value; and
providing information about a predicted defect-causing factor based on a comparison result between the measurement value and the simulation value.

2. The method of claim 1, wherein the input data comprises process data of the semiconductor device.

3. The method of claim 1, further comprising:
obtaining the measurement value from an image of the semiconductor device; and
obtaining the simulation value from an image of the virtual semiconductor device.

4. The method of claim 1, wherein the comparing of the measurement value with the simulation value comprises calculating similarity between the measurement value and the simulation value by using an artificial neural network.

5. The method of claim 1, wherein the obtaining of the simulation value comprises modeling the manufacturing process.

6. The method of claim 1, wherein the obtaining of the simulation value is performed by using technology computer-aided design (TCAD).

7. The method of claim 1, wherein the obtaining of the measurement value comprises measuring the semiconductor device by using at least one of:
a method of optically inspecting the semiconductor device; and
a method of inspecting the semiconductor device by using a transmission electron microscope (TEM).

8. A method of manufacturing a semiconductor process, the method comprising:
obtaining a measurement value of a semiconductor device;
obtaining a simulation value of a virtual semiconductor device based on input data about a manufacturing process of the semiconductor device, wherein the input data comprises a suspected defect-causing factor; and
comparing the measurement value with the simulation value by comparing similarity between an image of the semiconductor device that provides the measurement value and an image of the virtual semiconductor device that provides the simulation value.

9. The method of claim 8, wherein the comparing of the measurement value with the simulation value is performed using a convolutional neural network (CNN).

10. The method of claim 8, wherein the obtaining of the simulation value is performed by changing at least one of physical characteristics of the suspected defect-causing factor and a time of occurrence of the suspected defect-causing factor.

11. The method of claim 10, wherein the physical characteristics of the suspected defect-causing factor comprise at least one of a shape of the suspected defect-causing factor, a size of the suspected defect-causing factor, a position of the suspected defect-causing factor, and a property of the suspected defect-causing factor.

12. The method of claim 8, wherein the comparing of the measurement value with the simulation value is performed by aligning similarities between a plurality of simulation values and the measurement value in descending order.

13. The method of claim 8, wherein the obtaining of the measurement value and the obtaining of the simulation value comprise detecting a boundary of the image of the measurement value and the image of the simulation value.

14. The method of claim 13, wherein the detecting of the boundary is performed using a gradient filter.

15. The method of claim 8, wherein the measurement value comprises at least one of a plan view, a front view, a side view and a cross-sectional view of the semiconductor device, and
the simulation value comprises at least one of a plan view, a front view, a side view and a cross-sectional view of the virtual semiconductor device.

16. The method of claim 8, wherein the defect-causing factor comprises particles.

17. A method of manufacturing a semiconductor device, the method comprising:
preparing a wafer;
forming a semiconductor device by performing a semiconductor process on the wafer; and
inspecting the semiconductor process,
wherein the inspecting of the semiconductor process comprises:
obtaining a measurement value of the semiconductor device;
obtaining a simulation value of a virtual semiconductor device based on input data about a manufacturing process of the semiconductor device, wherein the input data comprises a suspected defect-causing factor;
comparing the measurement value with the simulation value; and
providing information about a predicted defect-causing factor based on a comparison result between the measurement value and the simulation value.

18. The method of claim 17, wherein the comparing of the measurement value with the simulation value comprises calculating similarity between an image of the measurement value and an image of the simulation value.

19. The method of claim 17, wherein, when the semiconductor device comprises a defect, the inspecting of the semiconductor process is performed.

20. The method of claim 17, further comprising:
obtaining the measurement value from a plan view and a cross-sectional view of the semiconductor device; and
obtaining the simulation value from a plan view and a cross-sectional view of the virtual semiconductor device.
